(19) United States
(12) Patent Application Publication
(10) Pub. No.: US 2023/0007905 A1
(43) Pub. Date: Jan. 12, 2023

(54) APPARATUS AND METHOD FOR PIERCING A SOFT TISSUE CANAL IN A BODY PART AND IMPLANT FOR INSERTION INTO A SOFT TISSUE CANAL IN A BODY PART

(71) Applicant: MEDARTIS HOLDING AG, Basel (CH)

(72) Inventors: Kurt TSCHOPP, Liestal (CH); Andreas MULLIS, Hemmiken (CH)

(21) Appl. No.: 17/783,568

(22) PCT Filed: Dec. 10, 2019
(86) PCT No.: PCT/EP2019/084344

§ 371 Date: Jun. 8, 2022

(51) Int. Cl. A61B 17/3213; A61B 17/34

(52) U.S. Cl. CPC A61B 17/3213; A61B 17/3468; A61B 2017/248

(57) ABSTRACT

Snoring and sleep apnea are conditions associated with a narrowing of the airway. A device (10) and a method for piercing a soft tissue channel in the pharynx and an implant for insertion into this soft tissue channel are disclosed, with which these symptoms can be treated. The device according to the invention comprises a handle (12) and a blade (11) with a tip (16), which are connected to each other via an interface (13). In this process, a channel is pierced through the device (10), the shape of which is designed in connection with the shape of the implant in such a way that the narrowing of the airways is optimally treated.

The invention relates to a device and a method for piercing a soft tissue channel in a body part as well as an implant for insertion into a soft tissue channel with the features of the generic term of the independent patent claims.

Sleep apnea is a typical condition among middle-aged people, the prevalence of which is higher in men than in women. People with sleep apnea, especially with obstructive sleep apnea syndrome (OSAS), suffer from slackening of the muscles in the upper air-ways. The resulting relaxation of the upper airways, especially during sleep, subsequently leads to a narrowing of the respiratory tract. The narrowing of the respiratory tract can lead to temporary respiratory arrest and/or a drop in the oxygen concentration in the blood with corresponding secondary diseases and damage.

Snoring is based on a similar narrowing of the airways. OSAS is therefore often accompanied by heavy snoring. However, snoring can also occur independently of OSAS.

The therapy of OSAS and snoring can be performed using implants. The focus is on stabilizing the slackened musculature to prevent fluttering of the palate and obstruction of the airways. An example of such an implant is disclosed in EP 2204148.

Such implants are currently implanted using conventional scalpets and blades. This requires a channel to be pierced into which the implant can be inserted.

Conventional blades have a fixed shape which essentially dictates the size and geometry of a piercing channel. It is known from prior art that implants for the treatment of OSAS and snoring have an angled shape. Therefore, an instrument used to pierce a soft tissue channel intended to receive such an implant must presently have a correspondingly curved shape.

Various surgical instruments with curved shapes are known in the prior art.

CN 202776720 U discloses a medical scalpel in which the front part of the blade is curved at an angle of 90 to 135° relative to the main axis of the instrument. The front of the blade thereby has a cutting surface.

DE 20 2007 008 229 U1 discloses a detachable curved sinusoidal blade. This has an outer cutting unit and an inner blade unit.

WO 2004/084709 A2 discloses a shaft and blade that can be used to implant an implant into the tongue or a pharyngeal canal. The blade serves for insertion of the shaft into the tissue and can be withdrawn through the shaft. The shaft is elastic. However, the prior art is limited to instruments that can cut or pierce a channel with the same shape as that of the instrument. This leads to problems especially if the canal needs to have a non-uniform curvature, since the curvature in the scalpel tip dictates the curvature of the entire canal. Such a canal can therefore not be pierced with the known instruments. It is therefore the object of the present invention to avoid the disadvantages of the prior art, and in particular to provide a device for piercing a soft tissue channel in a body part which has a shape suitable for implants of various shapes.

According to the invention, these and other tasks are accomplished with a device and a method for piercing a soft tissue channel in a body part as well as an implant for insertion into a soft tissue channel in a body part with the features of the independent claim.

The device according to the invention for piercing a soft tissue canal in a body part, in particular in the pharynx, has a blade. The shape, size and material of the blade are selected so that the blade deforms when the soft tissue canal is pierced, in particular in the hard and/or soft palate. The blade deforms in such a way that the blade forms a soft tissue channel that follows the shape of the palate. The shape and geometry of the soft tissue channel does essentially not correspond to the shape of the blade in the undeformed state.

In particular, the device according to the invention allows to pierce a soft tissue channel without tearing the soft tissue.

In an alternative embodiment, the blade is formed so that it does not kink when piercing the soft tissue canal and is deformed into a shape that forms the soft tissue canal due to the structure of the soft tissue canal.

In a preferred embodiment, the blade is connected or connectable to a handle via an interface.

In an alternative embodiment, the blade is integrally formed with the handle.

In a preferred embodiment, the blade is made of a material having a bending strain of 0.4 to 20%, preferably 0.5 to 10%, more preferably 0.8 to 2%.

In a preferred embodiment, the blade is configured such that a cross-sectional area of the blade, measured in a plane perpendicular to the longitudinal axis of the blade, is from 0.025 to 300 mm2, preferably 0.5 to 10 mm2, more preferably 1 to 2 mm2. In a preferred embodiment, the blade is designed in such a way that the cross-sectional area of the blade is convex and/or has straight boundary surfaces. In particular, boundary surfaces can be arranged at right angles to one another and connected to one another via curved surfaces, the convex boundary surfaces having a radius of curvature of 1 to 10000 mm. In another preferred embodiment, the convex boundary surfaces have a radius of curvature of 2 to 1000 mm. In another preferred embodiment, the convex boundary surfaces have a radius of curvature 10 to 200 mm. In a preferred embodiment, at least one surface of the blade is designed such that an implant can be slid along the surface. The surface has an arithmetic average roughness of 0.001 to 100 μm, preferably 0.05 to 50 μm, particularly preferably 0.1 to 0.2 μm. In accordance with the common definition, the mean roughness value is the arithmetic mean roughness value corresponding to the mean value of all deviations from a reference distance. The mean value of the height profile is taken as the reference distance. The arithmetic mean roughness value can be measured by scanning a surface and creating a one- or two-dimensional height profile. The mean value of the height of the profile can be determined or calculated so that the deviations from the mean value can be summed or integrated. Division by the length of the reference line or the area of the reference surface then gives the arithmetic average roughness value.

In a preferred embodiment, the blade has an extended length of 5 to 300 mm, preferably 10 to 100 mm, more preferably 75 to 85 mm. In particular, the blade may have a width of 5 to 25 mm, preferably 10 to 20 mm, more preferably 15 to 17 mm.

The handle can have a thickness of 0.5 to 5.0 mm, preferably 1.0 to 4 mm, particularly preferably 1.5 to 2.5 mm. Its height is in particular between 10 and 35 mm, preferably between 15 and 25 mm, particularly preferably between 18 and 23 mm. Its length is in particular 100 to 200 mm, preferably 125 to 175 mm, particularly preferably 140 to 160 mm.

In the inserted state, i.e. when the blade is in operative connection with a corresponding counterpart on the handle via a coupling such as is used with commercially available scalpels, the ratio of handle length to blade length is in particular between 1:0.1 and 1:1, preferably between 1:0.3 and 1:0.8, particularly preferably from 1:0.45 to 1:0.55.

The width of the device is in particular between 5 and 20 mm, preferably between 5 and 10 mm, particularly preferably between 6 and 8 mm.

In a particularly preferred embodiment, the handle has a width of 7.5 mm. In addition, the handle can in particular have a coupling region with a width of 2.36 mm, which can be connected to the handle via a region with a width of 4 mm. In a preferred embodiment, the blade is substantially partially curved along a longitudinal axis. The blade has a radius of curvature of 1 to 10000 mm, preferably 2 to 1000 mm, particularly preferably 10 to 200 mm.

In a preferred embodiment, the blade is made of metal, in particular steel.

In a preferred embodiment, the blade has a tip on the opposite side of the handle. The tip further comprises a cutting edge formed over an angle of 10 to 80°. In a further embodiment, the tip has a cutting edge formed over an angle of 45 to 65°. Particularly preferably, the angle has a value of 60°. In particular, a cutting edge according to DIN 58298 is used and its cutting ability is tested according to DIN 58298.

The implant according to the invention for insertion into a soft tissue canal in a body part, in particular in the pharyngeal cavity, comprises a shaped piece which is designed in such a way that, due to an internal tension in the shaped piece, the stability of the body part with the implant received in the soft tissue canal is increased compared to that of the stability of the body part without the shaped piece.

The implant is particularly suitable to be grasped by surgical micro forceps, allowing easy insertion of the implant into the soft tissue channel via the guide instrument. This eliminates the need to provide the implant with a coupling for connection to the guide instrument.

The method according to the invention for piercing a soft tissue canal in a body part, in particular a soft tissue canal in the pharynx, in particular in the hard and/or soft palate, comprises the steps described below. It comprises piercing a soft tissue channel with a device having a blade which deforms during piercing. In a preferred embodiment of the method, an embodiment of the device according to the invention described above is used. The blade forms a soft tissue channel whose shape and geometry does not substantially correspond to the shape of the blade in the undeformed state.

Particularly preferably, the method according to the invention further comprises the insertion of an implant, in particular an implant according to an embodiment according to the invention, into the soft tissue canal, in particular into a soft tissue canal of the pharynx. The insertion takes place along a surface of the blade.

Optionally, the method according to the invention comprises removing the device from the soft tissue canal and inserting a guide instrument into the soft tissue canal. The insertion of an implant, in particular an implant according to a preferred embodiment of the invention, into the soft tissue canal, in particular into the soft tissue canal of the pharyngeal cavity, takes place in this preferred embodiment along the surface of the guide instrument.

Optionally, the method according to the invention comprises removing the device from the soft tissue canal and inserting an implant into the soft tissue canal, in particular into the soft tissue canal of the pharynx. In particular, an implant according to a preferred embodiment of the invention can be used.

Preferably, the method according to the invention comprises inserting, in particular pushing, an implant into the soft tissue canal. In this process, the implant has a shaped piece which is designed in such a way that the shape of the shaped piece does not correspond exactly to the shape and geometry of the soft tissue canal.

The method according to the invention, in particular the use of a device according to the invention for piercing the soft tissue canal, makes it possible to insert the implant in such a way that it lies in the soft tissue canal with little tension, preferably essentially without tension. For this purpose, in particular, an angle of the soft palate can be estimated and a suitable implant can be selected. For example, the angle of the soft palate could be estimated to be 40°, so that an implant with an angle of 40° would be selected accordingly. In particular, when the device is inserted, the soft palate straightens, since the device cannot pierce at an angle, and a canal is formed in it. After insertion of the implant, which has been selected in shape to match the anatomy of the palate, and retraction of the device, the soft palate largely returns to the neutral position.

It is of course conceivable that a deviation from the original neutral position occurs due to an inaccurate determination of the palatal angle. For example, the actual angle of the soft palate in the above example could have been 41°. In this case, the palate could be at an angle of 40.5° after removal of the device, for example, and the implant could have a small tension. It is particularly advantageous to keep the deviation between the estimated and actual palatal angle as small as possible. A movement of the soft palate out of this neutral position leads to a small deformation of the implant which tries to bring the tissue back into the neutral position.

Therefore, the aim of treatment is to influence the movement of the soft palate during breathing and snoring.

Preferably, the method according to the invention comprises elastic deformation of the implant after piercing the soft tissue canal and inserting the implant into the soft tissue canal. In this process, the soft tissue canal is at least partially filled by the implant.

The invention is explained in more detail below with reference to the figures and embodiment examples, showing:

FIG. 1a: the device for piercing a soft tissue channel in a body part from a laterally elevated perspective.

FIG. 1b: the device for piercing a soft tissue channel in a body part from a lateral perspective.

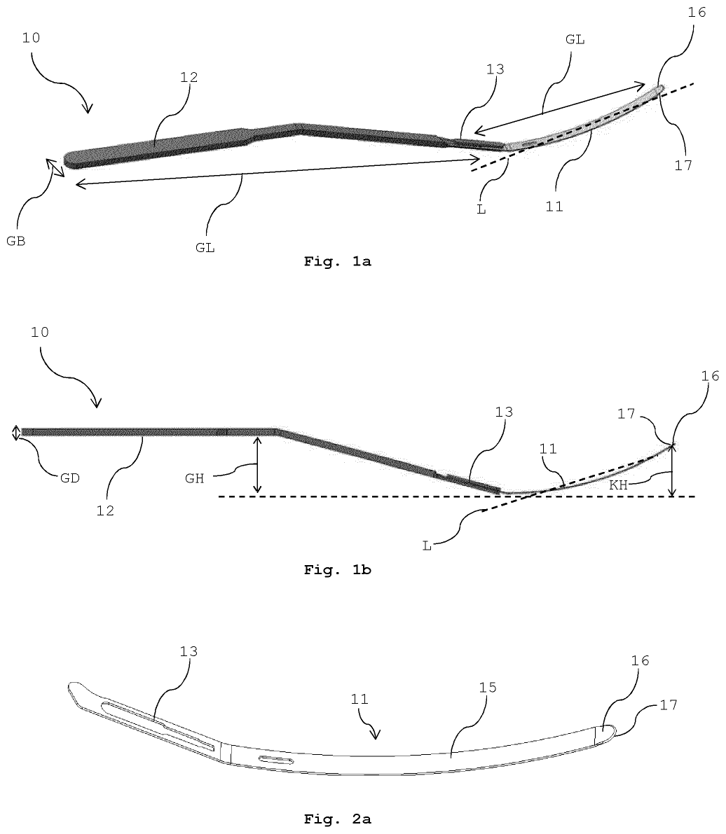

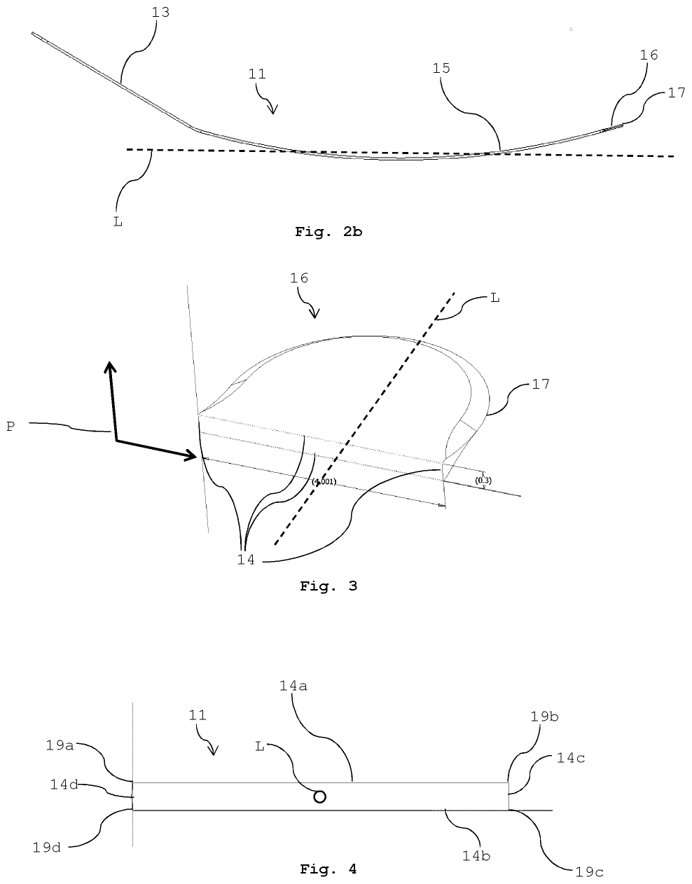

FIG. 2a: a blade for use as part of a device according to the invention.

FIG. 2b: the blade from FIG. 2a in a side view.

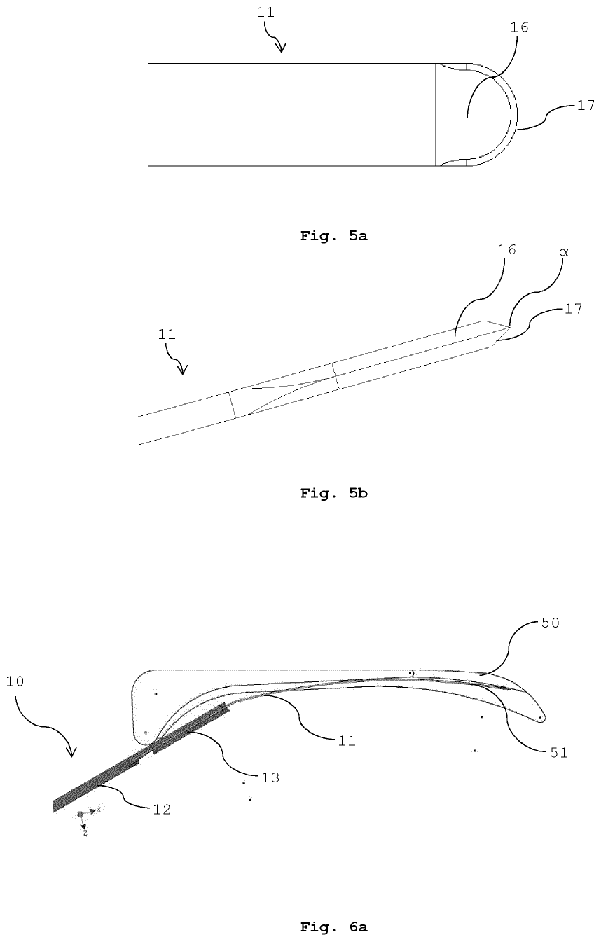

FIG. 3: a perspective view of the tip of the blade from FIG. 2a.

FIG. 4: a cross section of a blade of an embodiment according to the invention.

FIG. 5a: a top view of the tip of the blade according to FIG. 2a.

FIG. 5b: a side view of the tip of the blade of FIG. 5a.

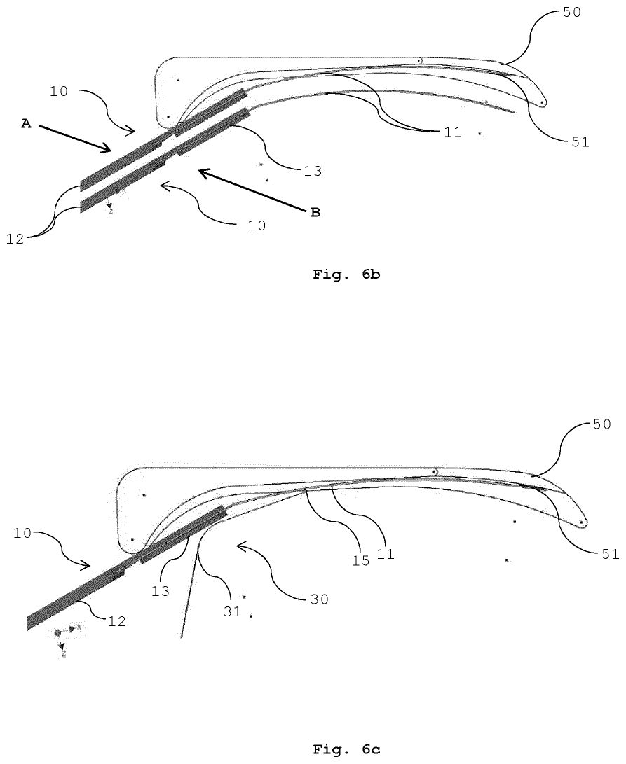

FIG. 6a. a device according to the invention when piercing a soft part of a patient.

FIG. 6b: a device according to the invention in the deformed and undeformed state.

FIG. 6c: a device according to the invention with an implant.

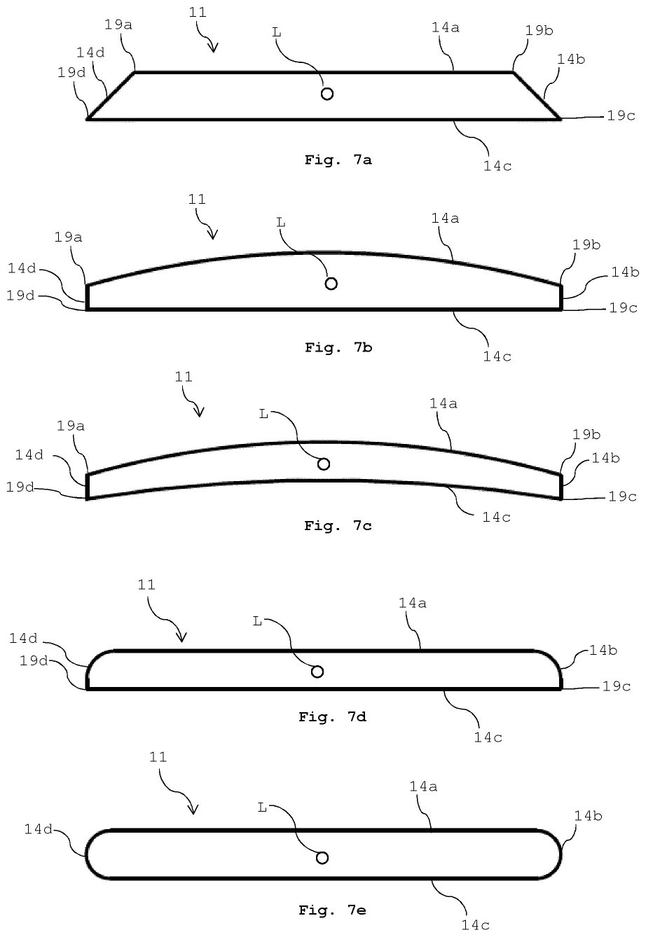

FIGS. 7a-7e: show cross sections of alternative embodiments of a blade.

FIG. 1a shows a preferred embodiment of the device 10 according to the invention for piercing a soft tissue channel in a body part. This comprises a handle 12 to which a blade 11 is connected or connectable via an interface 13. The interface corresponds to a coupling such as is present in commercially available scalpels. On the opposite side of the handle 12, the blade 11 comprises a tip 16 with a cutting edge 17. The blade 11 in FIG. 1a is partially curved along the longitudinal axis L, which corresponds to a preferred embodiment of the device according to the invention. In the version shown here, it consists of spring steel 1.4310.

FIG. 1b shows essentially the same device as FIG. 1a, but from a different perspective. In particular, the curvature of the blade is better visible in this perspective.

In the device shown in FIGS. 1a and 1b, the handle 12 has a handle length GL of 152 mm, a handle height GH of 21 mm, a handle width GB of 7.5 mm, and a handle thickness GD of 2 mm. The blade 11 has a length KL of 79 mm and a height KH of 16 mm. When the blade 11 is inserted into the handle 12, the length ratio of handle 12 to blade 11 is 1:0.52.

FIG. 2a shows a blade 11 as it can be used in a device according to the invention. The blade comprises an interface 13 with which it can be reversibly fixed to a handle (not shown). In the present example, this is an interface such as is also present in commercially available scalpels. The tip 16 of the blade 11 comprises a cutting edge 17, which in the present example is semicircular and does not enclose a tip with an angle. It merges flush with the remaining part of the blade 11 and its surface 15. The surface 15 has, for example, a center roughness value of 10 μm. FIG. 2b shows essentially the same blade 11 as FIG. 2a in a side view.

FIG. 3 shows the tip 16 of the blade 11 in detail. The tip 16 is located on the opposite side of the handle with respect to the longitudinal axis L (not shown). The tip has a cutting edge 17 which is symmetrical to the longitudinal axis. However, it would also be conceivable to form the cutting edge asymmetrically with respect to the longitudinal axis L. Boundary surfaces 14 form the cross section of the blade 11. FIG. 4 shows a cross-section of the blade 11 in a plane perpendicular to the longitudinal axis L, designated P in FIG. 3, of the device 10 according to the invention. The boundary surfaces 14a, 14b, 14c, 14d of the blade in the embodiment shown here are straight and are at an angle 19a, 19b, 19c, 19d of 90° to each other. FIG. 5a shows the tip area of the blade 11 in a bird's eye view. The embodiment shown corresponds essentially to the embodiment shown in FIG. 2a and comprises a circular tip 16 with a cutting edge 17. Alternatively, however, pointed, elliptical or conical tips are also conceivable, each of which can be symmetrical or asymmetrical. FIG. 5b shows a side view of a tip region of the blade 11 of FIG. 5a. The cutting edge 17 also extends laterally over a portion of the blade 11. In particular, the blade 11 in the embodiment shown here comprises a point 16 with a ground angle α, which here is 60°. Symmetrical or asymmetrical designs as well as other angles are also possible.

FIGS. 6a-6c schematically show the sequence of a preferred method according to the invention. First, a device 10 according to the invention, which can be held by the operator via the handle 12, is pierced into a body part 50. The blade 11 deforms during the piercing, so that the shape of the channel 51 does essentially not correspond to the original shape of the blade. FIG. 6b shows a comparison of a device 10 implanted in the body part 50 of a patient (A) and a device 10 outside the body part in an undeformed state (B). The blade 11 of the device 10 was deformed during the piercing of the channel 51 (A) and has a smaller curvature compared to the device 10 outside the body part 50 (B).

FIG. 6c shows the insertion of an implant 30 comprising a shaped piece 31. This is pushed into the canal 51 along the surface 15 of the blade 11. The shape of the implant 30 was selected according to the patient's anatomy in order to achieve a desired return to the neutral position in the inserted state in the event of a deviation from the neutral position. FIG. 7a shows a cross-section, similar to that of FIG. 4, of a blade 11 in an alternative embodiment. The upper boundary surface 14a and the lower boundary surface 14c are straight and parallel to each other. The boundary surfaces 14b, 14d are also straight, but converge towards the upper boundary surface 14a. Thus the cross-section of the blade 11 as a whole forms a trapezoid. The angles 19a, 19b between the lateral boundary surfaces 14b, 14d and the upper boundary surface are obtuse, while the angles between the lateral boundary surfaces 14b, 14d and the upper boundary surface 14a are acute.

FIG. 7b shows a cross-section of a blade 11 in an alternative embodiment. The blade 11 is similar to that of FIG. 4, but the upper boundary surface 14a is convex, whereby the angles between this boundary surface 14a and the lateral boundary surfaces 14b, 14d are slightly greater than 90°. FIG. 7c shows a cross-section of an alternative embodiment of a blade 11. The lateral boundary surfaces 14b, 14d are straight and parallel. The upper boundary surface 14a is curved and convex in shape, while the lower boundary surface 14c is also curved but concave in shape. As a result, the cross-section as a whole has the shape of a curved band. Because the curvature of the lower boundary surface 14c is smaller than that of the upper boundary surface 14a, the thickness of the blade decreases toward the ends with the lateral boundary surfaces 14b, 14d. FIG. 7d shows a cross-section of another alternative embodiment of a blade 11. This blade is similar to the blade of FIG. 4. However, the lateral boundary surfaces 14b, 14d are connected to each other via rounded areas, so that the blade has angles 19c, 19d only at the lower boundary surface 14c. FIG. 7e shows a cross-section of a further alternative embodiment of a blade 11, which is similar to the blade of FIG. 7d. However, angled boundary surfaces are completely absent because the lower boundary surface 14c is also connected to the lateral boundary surfaces 14b, 14d via rounded areas.

1.-14. (canceled)

15. A device for piercing a soft tissue channel in a body part, comprising a blade, wherein the shape, size and material of the blade are selected in such a way that the blade deforms when the soft tissue channel is pierced, in such a way that the blade forms a soft tissue channel which is not straight and the shape and geometry of which does not substantially correspond to the shape of the blade in the undeformed state.

16. The device according to claim 15, wherein the blade is formed such that the blade does not kink when piercing the soft tissue channel and the blade is deformed into a shape that forms the soft tissue channel due to the structure of the soft tissue channel.

17. The device according to claim 15, wherein the blade is connected or connectable to a handle via an interface.

18. The device according to claim 17, wherein the blade is integrally formed with the handle.

19. The device according to claim 15, wherein the blade is made of a material having a bending strain of 0.4 to 20%.

20. The device according to claim 15, wherein a cross-sectional area of the blade measured in a plane perpendicular to a longitudinal axis is between 0.025 and 300 mm².

21. The device according to claim 15, wherein the cross-sectional surface of the blade is convex and/or formed of straight boundary surfaces, wherein the convex boundary surfaces have a radius of curvature of 1 to 10,000 mm.

22. The device according to claim 15, wherein at least one surface of the blade is designed such that an implant can be displaced along the surface and the surface has an arithmetic average roughness Ra of 0.001 to 100 μm.

23. The device according to claim 15, wherein the blade has an stretched length of from 5 to 300 mm.

24. The device according to claim 20, wherein the blade is substantially partially curved along a longitudinal axis and the blade has a radius of curvature of from 1 to 10000 mm.

25. The device according to claim 15, wherein the blade is made of metal.

26. The device according to claim 17, wherein the blade has a tip on the opposite side of the handle and the tip has a cutting edge, wherein the cutting edge is formed over an angle of 10 to 80°.

27. An implant for insertion in a soft tissue canal in a body part, comprising a shaped piece, wherein the shaped piece is designed in such a way that the stability of the body part with the implant accommodated in the soft part canal is increased by an internal tension in the shaped piece compared to the stability of the body part without shaped piece.

28. A method for treating and for piercing a soft tissue channel in a body part, comprising the step:
piercing a soft tissue channel with a device having a blade which deforms during the piercing wherein the blade forms a soft tissue channel whose shape and geometry does not substantially correspond to the shape of the blade in the undeformed state.
